(19) United States
(12) Patent Application Publication
(10) Pub. No.: US 2011/0104004 A1
(43) Pub. Date: May 5, 2011

(54) SPORT BALL SANITIZER

(76) Inventor: Gregory C. Bobbitt, Buda, TX (US)

(21) Appl. No.: 12/612,837

(22) Filed: Nov. 5, 2009

(51) Int. Cl. A61L 2/10; B01J 19/12

(52) U.S. Cl. 422/24; 422/186.3

(57) ABSTRACT

An apparatus for sanitizing a sport ball includes a container that receives the sport ball and at least one germicidal light source coupled to the container. The at least one germicidal light source emits germicidal wavelength light onto the sport ball within the container during operation of the apparatus. Alternatively, the apparatus includes a germicidal light-transparent receptacle that receives the sport ball and a plurality of germicidal light sources coupled to the germicidal light-transparent receptacle. The plurality of germicidal light sources emit germicidal wavelength light, at least in part through the germicidal light-transparent receptacle, onto each location on the surface of the ball for at least a predetermined intensity-time.

### FIELD OF THE INVENTION

The present invention relates in general to the field of sanitization equipment, and particularly to sanitization equipment for athletic gear.

### BACKGROUND OF THE INVENTION

When it comes to basketball—from high school through the pros—influenza is the hidden opponent on everyone's schedule. Influenza has been part of the sport's folklore since Game 5 of the 1997 NBA Finals, when the Chicago Bulls' Michael Jordan dragged himself out of bed to score 38 points against the Utah Jazz. This season, flu-like symptoms have thinned rosters in Atlanta, New York and Indianapolis. They had the Lakers' Kobe Bryant alternately vomiting, receiving intravenous fluids and scoring clutch baskets against the Cleveland Cavaliers last February. Local college teams have been hit just as hard, with a handful of USC and UCLA players struggling through games leading up to the Pacific 10 Conference tournament at Staples Center last March. “This year's been unbelievable,” said Wally Blase, the trainer for the Atlanta Hawks. “In the last month, we've had four guys get really sick and three guys miss time.”

Sneezing and coughing, the occasional virus that morphs into gastrointestinal distress or worse—athletes aren't the only ones who suffer. Like everyone else, they come across airborne and respiratory diseases in crowded places, but the nature of their sport can leave them especially vulnerable. Through the heart of flu season—roughly November to March—they face constant play and practice sharing a ball 9 other guys are sweating on and passing around. On that ball are germs and bacteria that breed illness.

Thus, there is a need for a quick non-fluid technique to sanitize sports balls, minimizing the risk of contact-borne illnesses for players.

### BRIEF SUMMARY OF INVENTION

In one aspect the present invention provides an apparatus for sanitizing a sport ball. The apparatus includes a container configured to receive the sport ball and at least one germicidal light source, coupled to the container. The at least one germicidal light source is configured to emit germicidal wavelength light onto the sport ball within the container during operation of the apparatus.

In another aspect, the present invention provides a method for sanitizing a sport ball. The method includes receiving the sport ball into a container and emitting germicidal wavelength light onto the sport ball within the container. The germicidal wavelength light is emitted by a germicidal light source coupled to the container.

In yet another aspect, the present invention provides an apparatus for sanitizing a sport ball. The apparatus includes a germicidal light-transparent receptacle configured to receive the sport ball and a plurality of germicidal light sources, coupled to the germicidal light-transparent receptacle. The plurality of germicidal light sources are configured to emit germicidal wavelength light, at least in part through the germicidal light-transparent receptacle, onto each location on the surface of the ball for at least a predetermined intensity-time.

### BRIEF DESCRIPTION OF THE DRAWINGS

FIG. 1 is a block diagram illustrating an end view of a sport ball sanitizer according to the present invention.

FIG. 2 is a block diagram illustrating a side view of the sport ball sanitizer of FIG. 1.

FIG. 3 is a block diagram illustrating an end view of a sport ball sanitizer according to an alternate embodiment of the present invention.

FIG. 4 is a block diagram illustrating a side view of the sport ball sanitizer of FIG. 3.

FIGS. 5 through 7 are side views of sport ball sanitizers according to alternate embodiments of the present invention.

FIG. 8 is a block diagram illustrating an end view of a sport ball sanitizer according to an alternate embodiment of the present invention.

FIG. 9 is a block diagram illustrating a side view of the sport ball sanitizer of FIG. 8.

FIG. 10 is a block diagram illustrating an end view of a sport ball sanitizer according to an alternate embodiment of the present invention.

FIG. 11 is a block diagram illustrating an end view of a sport ball sanitizer according to an alternate embodiment of the present invention.

FIG. 12 is a block diagram illustrating a side view of the sport ball sanitizer of FIG. 11.

### DETAILED DESCRIPTION OF THE INVENTION

Described herein are embodiments of an apparatus and method that employ a germicidal light source to sanitize a sport ball.

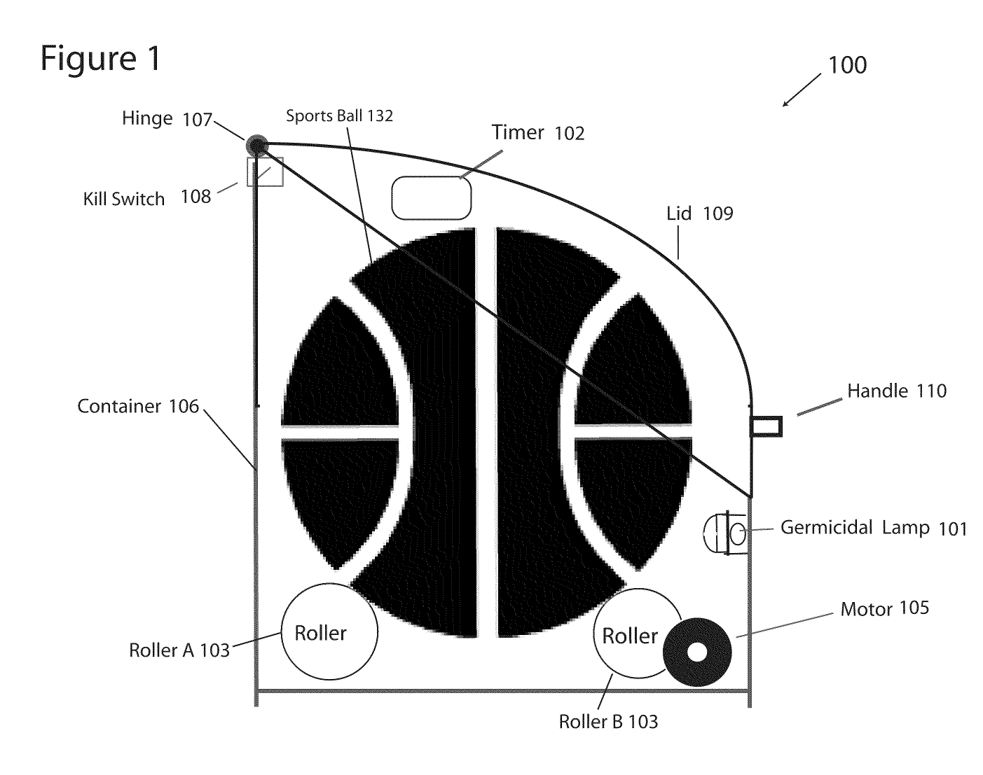

Referring now to FIG. 1, a block diagram illustrating an end view of a sport ball sanitizer 100 according to the present invention is shown. It should be noted that the Figures described herein are not drawn to scale. The ball sanitizer 100 includes a container 106. The container 106 encloses a germicidal light source 101. Advantageously, the germicidal light source 101 emits light at a germicidal wavelength in order to sanitize a sport ball 132 enclosed within the container 106 by to killing germs on its surface. The characteristics of the germicidal light source 101 according to various embodiments and its operation to sanitize a ball 132 will be described in more detail below. Preferably, the germicidal light source 101 has a minimal damaging effect upon the sport ball surface material. In embodiments in which the germicidal light may be harmful to humans or other non-germ life forms, the container 106 is constructed to prevent the germicidal light emitted by the germicidal light source 101 from escaping the container 106 when it is closed. In one embodiment, portions of the surfaces of the container 106 are covered with reflective material to reflect the germicidal light back on the surface of the ball 132 to increase the intensity of the germicidal light upon the ball 132.

The container 106 includes a lid 109, or door 109, coupled to a bottom portion by a hinge 107. The lid 109 includes a handle 110 that enables a user to open the lid 109 and insert a ball 132 to be sanitized. A kill switch 108 coupled to the container 106 is positioned such that when the lid 109 is opened, the kill switch 108 causes the germicidal light source 101 to cease emitting the germicidal light until the lid 109 is closed again in order to avoid exposing users to the germicidal light.

The container 106 encloses a plurality of rollers 103 upon which the ball 132 rests when inserted. The embodiment of FIG. 1 includes two rollers 103, denoted roller A and roller B. In one embodiment, the germicidal light source 101 is advantageously located near the lower portion of the container 106 nearest roller B so that for small diameter balls, the location of roller A 103 is capable of being adjusted toward roller B 103 to cause the smaller ball to also rest against roller B 103.

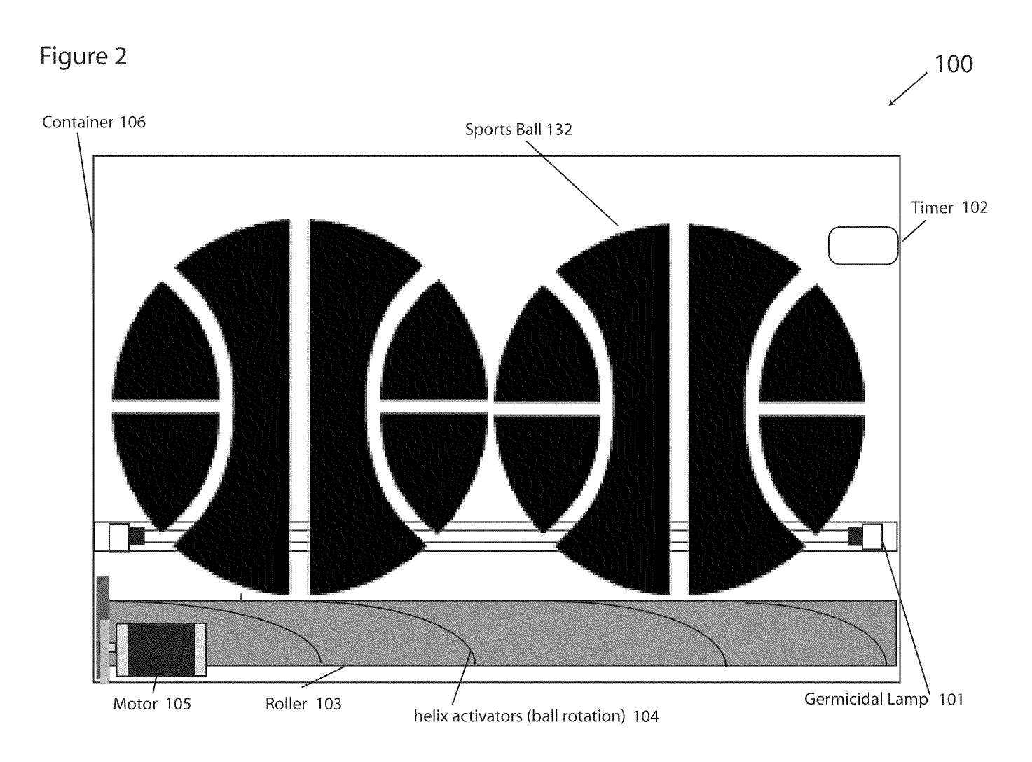

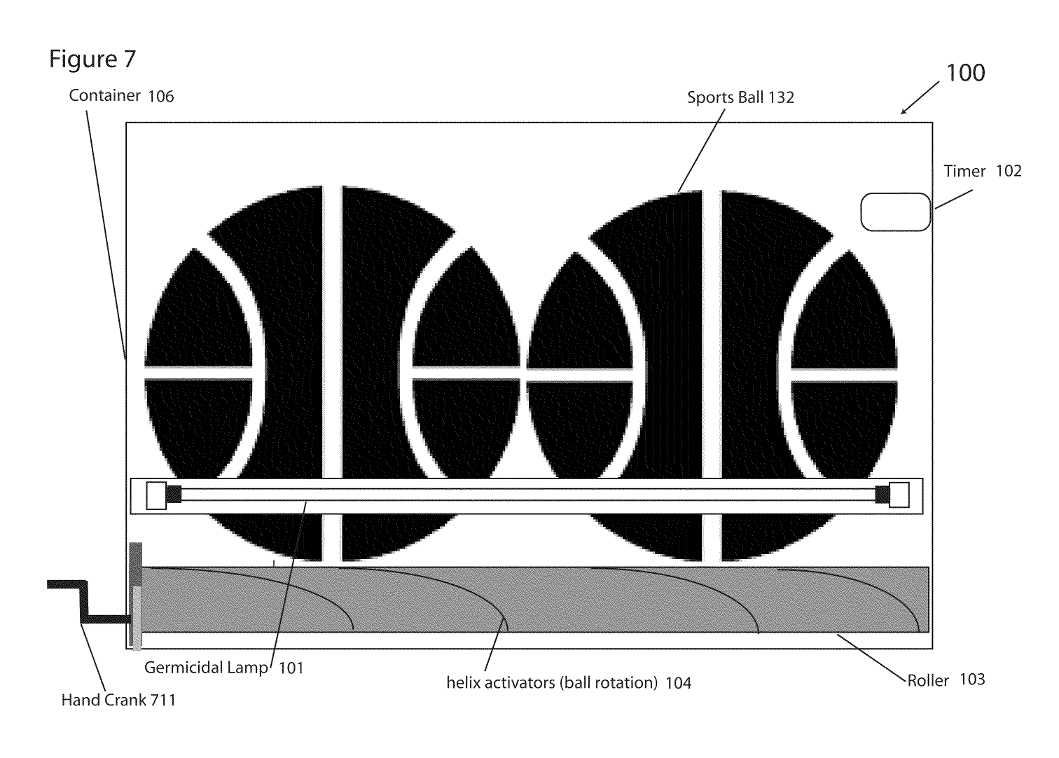

The container 106 also encloses a motor 105. The motor 105 is in communication with at least one of the rollers 103 (with roller B in the embodiment of FIG. 1) to cause the roller 103 to rotate when the motor 105 spins. Thus, during operation of the sport ball sanitizer 100, the motor 105 and rollers 103 work together to cause the ball 132 to rotate so that the entire surface of the ball 132 is exposed to the germicidal light emitted by the germicidal light source 101 to sanitize the ball 132. In one embodiment, the rollers 103 include an uneven surface, such as helix activators 104 (shown in FIG. 2) that causes the ball 132 to rotate in both a horizontal and vertical direction. The motor 105 may be any type of motor capable of causing the rollers 103 to rotate to cause the ball 132 to rotate. For example, the motor 105 may include an alternating current (AC) motor or a direct current (DC) motor. Additionally, a hand-crank 711 may be coupled to the roller A 103 to enable a human to rotate the roller A 103 (as shown in FIG. 7) to cause the ball 132 to rotate.

As mentioned above, the germicidal light source 101 emits light at a germicidal wavelength in order to sanitize a sport ball 132 enclosed within the container 106 by to killing germs on its surface. In one embodiment, the germicidal wavelength of the light emitted by the germicidal light source 101 is in less than 300 nanometers. More specifically, in one embodiment, the germicidal wavelength of the light emitted by the germicidal light source 101 is in a range between 240 and 280 nanometers. Yet more specifically, in one embodiment, the germicidal wavelength of the light emitted by the germicidal light source 101 is in 254.7 nanometers. In another embodiment, the germicidal wavelength of the light emitted by the germicidal light source 101 is in 253.7 nanometers. The germicidal light at these wavelengths has been observed to alter the DNA of germs such that the germ cannot effectively reproduce, thereby accomplishing a sanitizing effect with respect to human use of the ball 132. That is, the exposure of germs to these wavelengths of germicidal light may not actually kill the germs. Thus, it should be understood that the use of the term “kill” in the present context may refer not only to causing the germ to die, but also to effectively rendering the germ of being unable to effectively reproduce.

Although embodiments with particular wavelengths are described herein, it should be understood that the apparatus and method may employ germicidal light of other wavelengths that are effective in killing germs that are intended to be killed on the ball 132. Furthermore, as light of other wavelengths is discovered to have germicidal properties in the future, germicidal light sources 101 emitting light of these wavelengths may be incorporated as equivalents within the embodiments described herein.

The sport ball sanitizer 100 operates in a manner to expose the germs on the surface of the ball 132 to the germicidal light at a sufficient intensity, measured in milliwatts per square centimeter (mW/cm²), for a sufficient amount of time, measured in seconds. The amount of intensity-time (mW-sec/cm²) required to kill different germs varies. Table 1 lists approximate intensity-times required to achieve a 99% kill rate for the various above-listed common bacteria, viruses, yeasts, and molds, according to one researcher. Although a kill rate of 99% is described, it should be appreciated that the sport ball sanitizer 100 may be designed to accomplish a larger or smaller kill rate. The listed germs are provided as examples of target germs; however, the present invention is not limited to sanitizing a ball 132 with germs in the list provided, but instead may be adapted to kill any set of target germs that may be killed by a germicidal light source 101.

TABLE 1

| | intensity-time |
|---|---|
| Germ | (mW-sec/cm²) |
| Bacteria | |
| Bacillus anthracis | 8,700 |
| Corynebacterium diptheriae | 6,500 |
| Escherichia coli | 7,000 |
| Legionella pneumophila (Legionnaires | 3,800 |
| Disease) | |
| Leptospira interrogans (Infectious | 6,000 |
| Jaundice) | |
| Salmonella enteritidis | 7,600 |
| Salmonella typhosa (Typhoid Fever) | 6,000 |
| Shigella dysenteriae (Dysentery) | 4,200 |
| Streptococcus hemolyticus | 5,500 |
| Vibrio cholerae (Cholera) | 6,500 |
| Virus | |
| Bacteriophage (E. Coli) | 26,600 |
| Hepatitis virus | 8,000 |
| Influenza virus | 6,600 |
| Poliovirus | 21,000 |
| Rotavirus | 21,000 |
| Yeasts | |
| Brewer's Yeast | 6,600 |
| Baker's Yeast | 8,800 |
| Mold | |
| Aspergillus flavus | 60,000 |
| Mucor racemosus | 17,000 |
| Oospora lactis | 6,000 |
| Penicillium digitatum | 44,000 |

The intensity of the germicidal light to which the germs are exposed may be roughly approximated as inversely proportional to the square of the distance between the location of the germ on the ball 132 surface and the germicidal light source 101. Where the germicidal wavelength light from multiple sources (i.e., directly from multiple germicidal light sources 101 and/or germicidal light from one or more germicidal light sources 101 reflected by reflective material within the container 106) reaches a given germ, the intensity-time experienced by the germ is the sum of the multiple individual intensity-times. The sport ball sanitizer 100 operates in a manner to expose the germs on the ball 132 surface to the germicidal light at a sufficient intensity-time to kill the target germs. It is common for germicidal light sources to be rated according to the intensity generated at one meter from the germicidal light source. In one embodiment, the germicidal light source 101 is constructed to generate germicidal light one meter away with an intensity of 120 mW/cm². However, germicidal light sources 101 of other intensity ratings may be employed. In order to determine the intensity of the germicidal light at distances other than one meter, the one meter rating may be multiplied by an intensity factor associated with the actual distance. Table 2 lists intensity factors for various distances (in inches) from the germicidal light source 101.

TABLE 2

| distance | intensity |
|---|---|
| (inches) | factor |
| 0 | 354 |
| 1 | 127 |
| 2 | 69 |
| 4 | 32 |
| 6 | 20 |
| 8 | 14 |
| 10 | 14 |
| 15 | 6 |
| 20 | 4 |
| 25 | 3 |
| 30 | 2 |
| 35 | 1.4 |
| 39.97 | 1 |

To illustrate by way of example, assume the following: the target germ is influenza that requires 6,600 mW-sec/cm² to kill; the germicidal light source 101 is rated at 120 mW/cm² at one meter; the distance from the germicidal light source 101 to a given location on the surface of the ball 132 is six inches. Therefore, the intensity-time required to kill the target germ is: (6,600 mW-sec/cm²)/[(120 mW/cm²)*20], or approximately 2.75 seconds. In the example, the sport ball sanitizer 100 operates for some calculated time value that is sufficient to expose each location on the surface of the ball 132 to the germicidal light at a distance of six inches for at least 2.75 seconds. It is noted that the example is provided for illustration purposes and the embodiments are not limited to the example given, either in terms of the germicidal light source 101 rating, the distance from the germicidal light source 101 to the ball 132 surface, or the target germs. Furthermore, in most embodiments the sport ball sanitizer 100 operates to expose each location on the surface of the ball 132 to the germicidal light for an amount of time that significantly exceeds the calculated value in order to increase the likelihood of killing germs on the ball 132 surface. In one embodiment, the motor 105 continuously rotates the ball sufficiently slowly such that each location on the surface of the ball 132 is exposed to the germicidal light for at least the required intensity-time. In another embodiment, when operation begins, the ball 132 is allowed to remain stationary on the rollers 103 for a predetermined time, then the motor 105 rotates the ball to a different position and stops for a predetermined time, then the motor 105 rotates the ball to a different position and stops for a predetermined time, and so forth, such that each location on the surface of the ball 132 is exposed to the germicidal light for at least the required intensity-time.

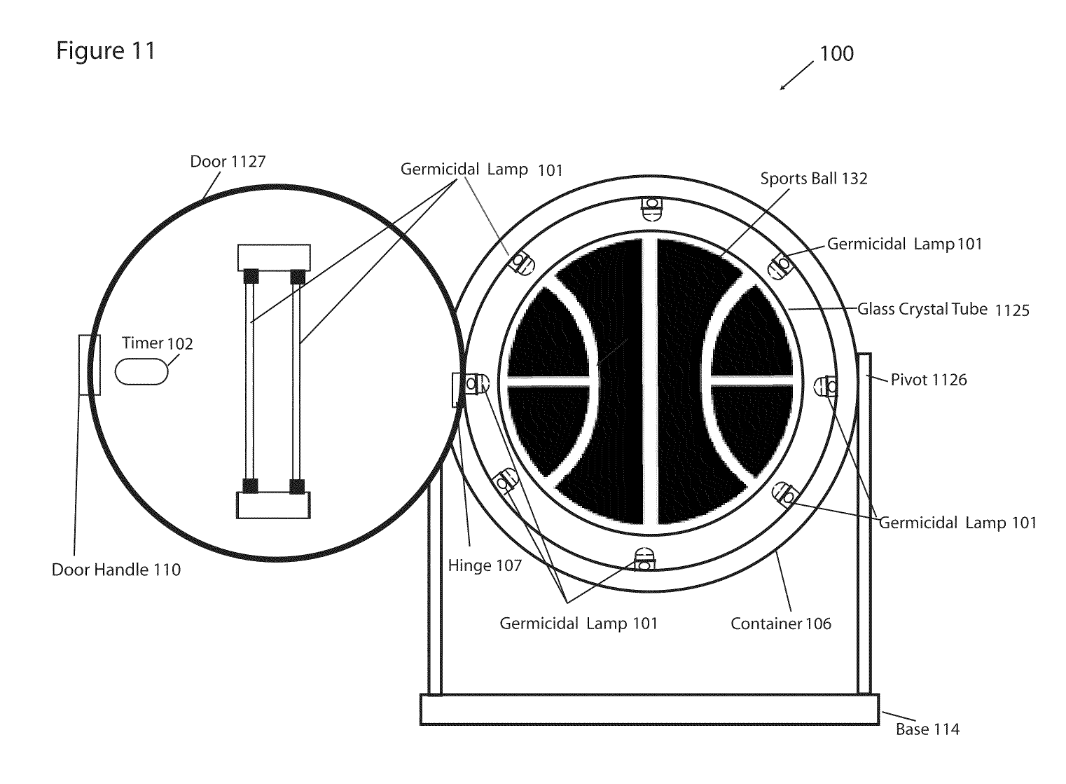

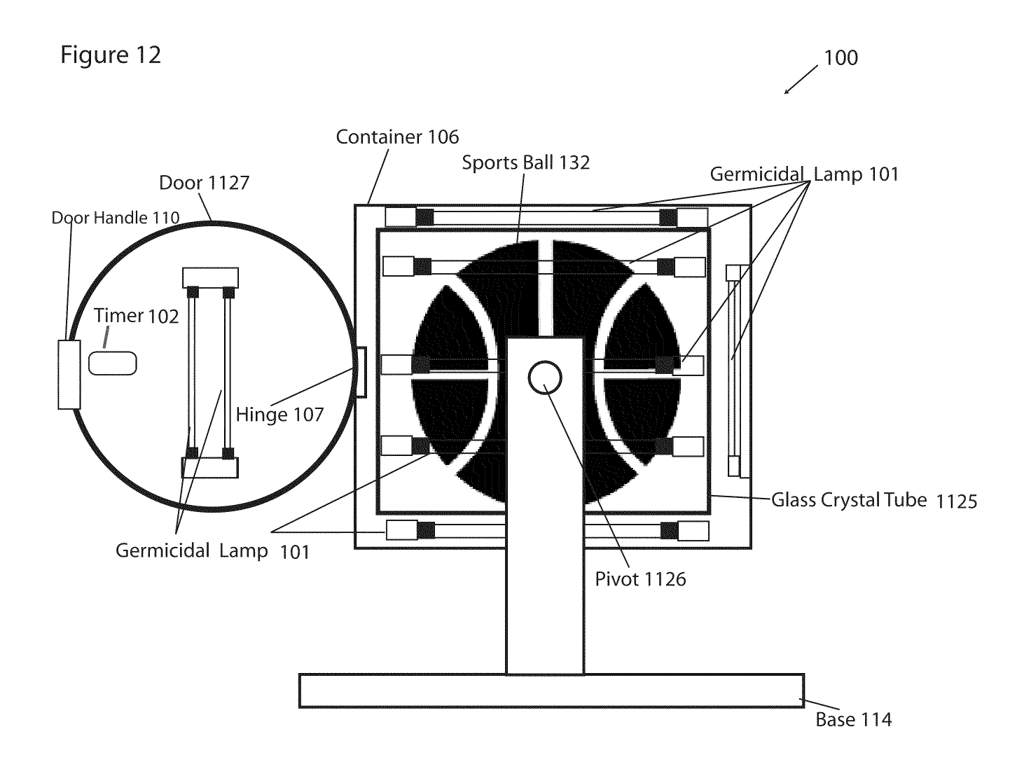

A timer 102 coupled to the container 106 controls the motor 105 and germicidal light source 101 to insure that the sport ball sanitizer 100 operates a sufficient amount of time such that each location on the surface of the ball 132 is exposed to the germicidal light for at least the required intensity-time. In one embodiment, the timer 102 displays a remaining amount of time until the ball 132 is sanitized. The calculated time value over which the sport ball sanitizer 100 operates (i.e., the germicidal light source(s) 101 emit the germicidal light and the motor 105 and rollers 103 operate to rotate the ball 132) to expose each location on the surface of the ball 132 for the required amount of time (2.75 seconds in the example above) to sanitize each location on the surface of the ball 132 is a function of various factors, such as, but not limited to: the number and location of the germicidal light source(s) 101; the shape and dimensions of ball 132; the rotation speed and directions of the ball 132 (if any, see embodiments of FIGS. 11 and 12 which do not rotate the ball 132); and the efficiency and location within the container 106 of the reflective material, if any.

Referring now to FIG. 2, a block diagram illustrating a side view of the sport ball sanitizer 100 of FIG. 1 is shown. As shown in FIG. 2, a plurality of balls 132 may be inserted into the sport ball sanitizer 100 and sanitized simultaneously. Two balls 132 are shown in FIG. 2. As shown in the embodiment of FIG. 2, the germicidal light source 101 is horizontally arranged within the container 106 to provide exposure of the entire width of the balls 132 to the germicidal light.

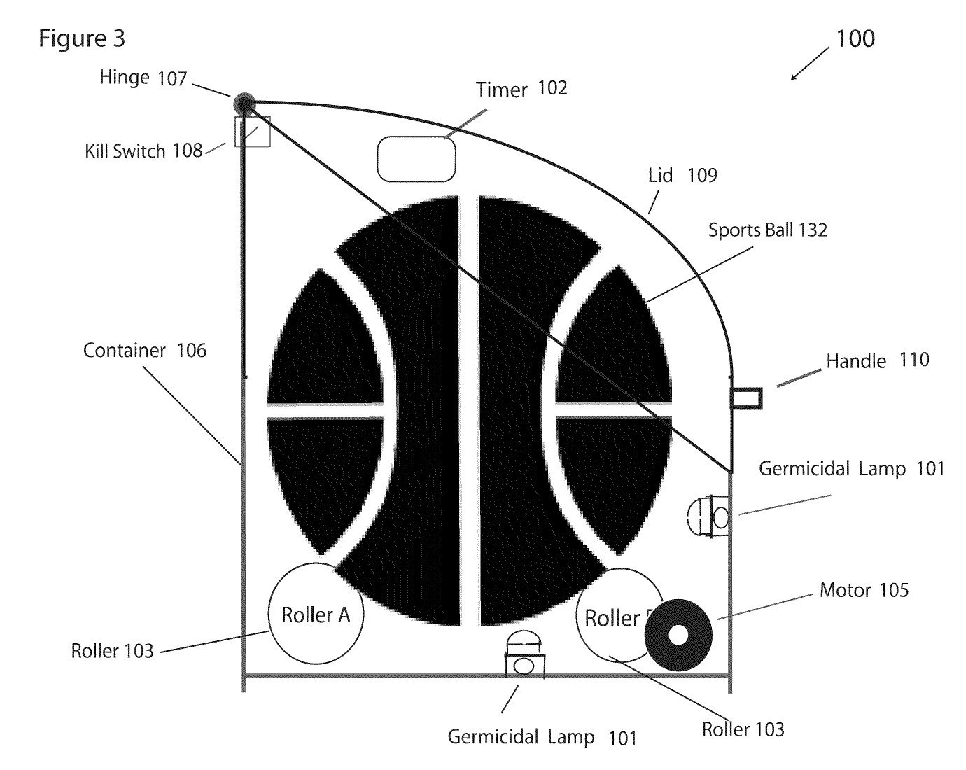

Referring now to FIG. 3, a block diagram illustrating an end view of a sport ball sanitizer 100 according to an alternate embodiment of the present invention is shown. The sport ball sanitizer 100 of FIG. 3 is similar to the sport ball sanitizer 100 of FIG. 1; however, the embodiment of FIG. 3 includes a plurality of germicidal light sources 101 in order to provide greater germicidal light intensity upon the surface of the ball 132. FIG. 3 illustrates an embodiment that includes two germicidal light sources 101, each oriented horizontally within the container 106.

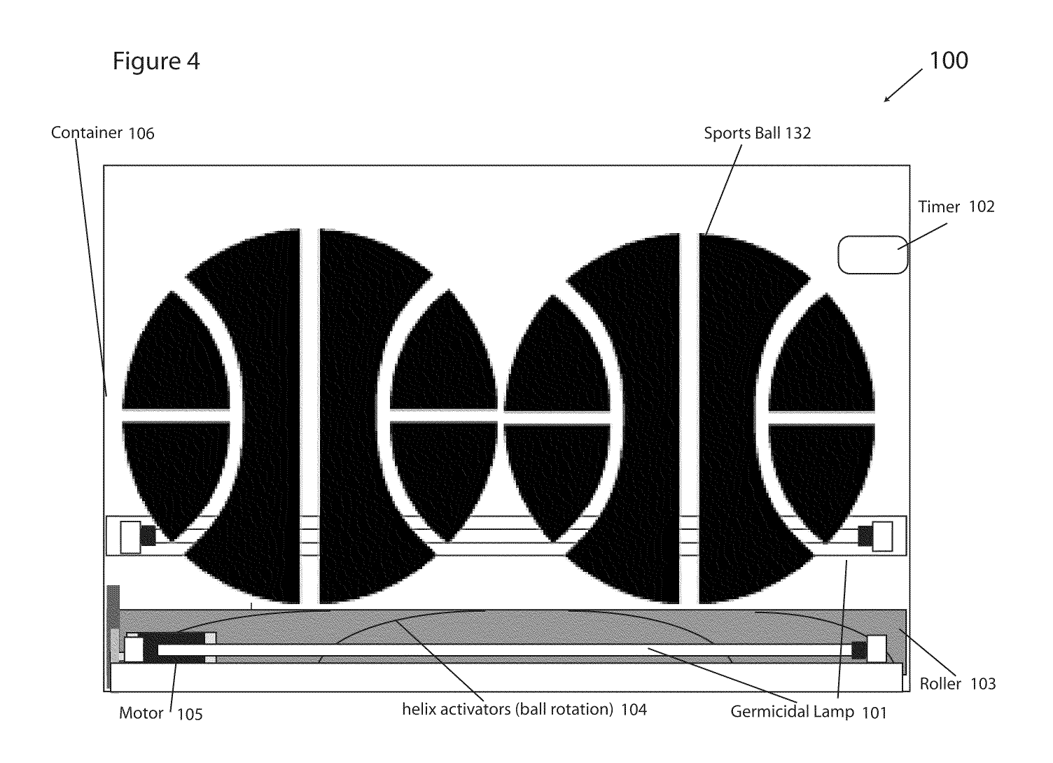

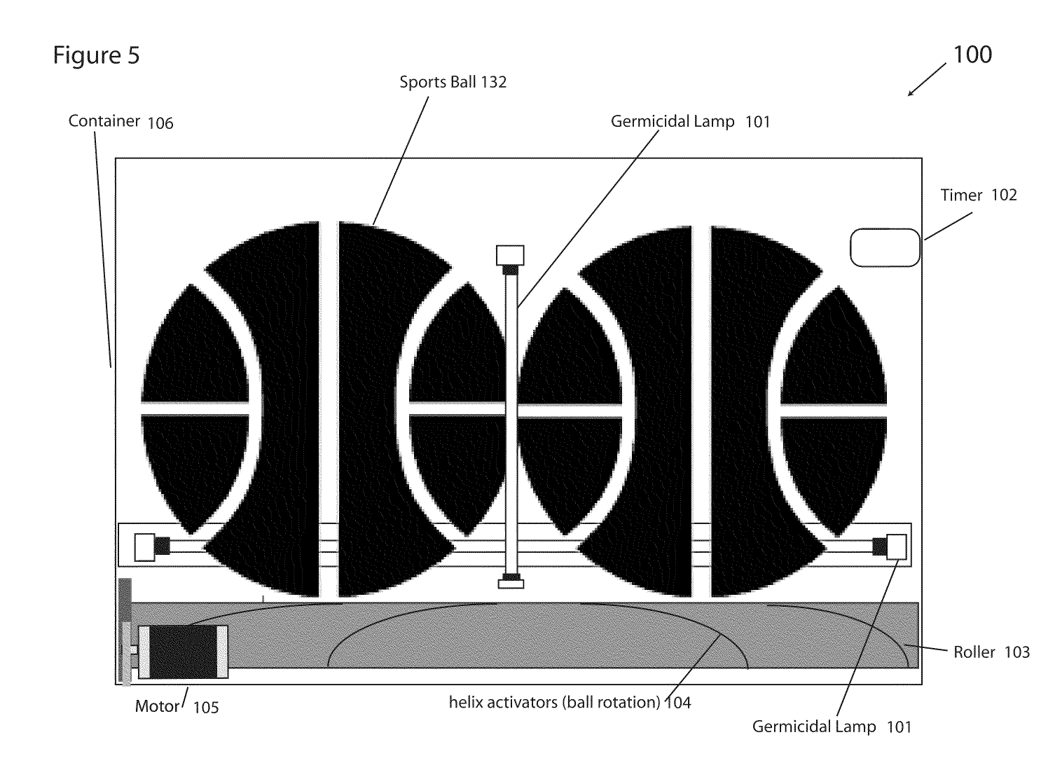

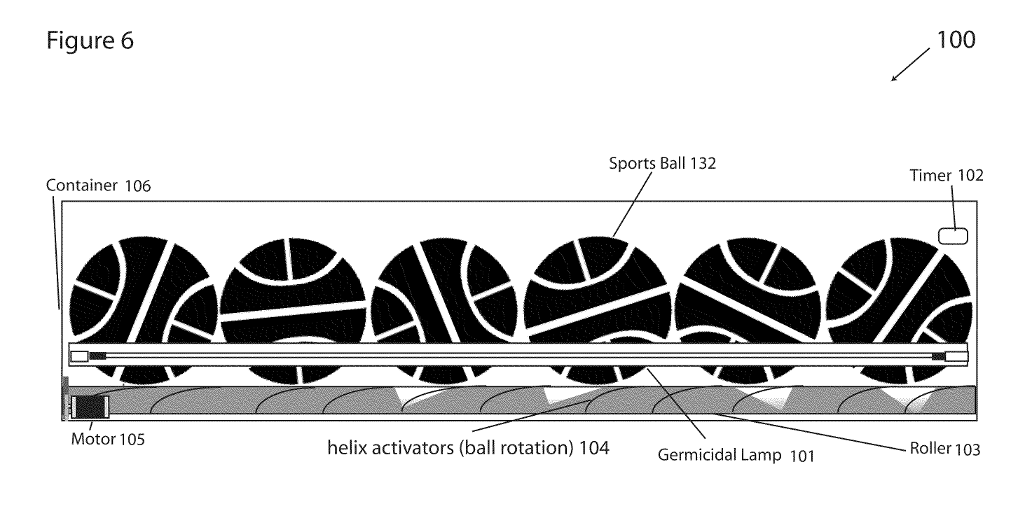

Referring now to FIG. 4, a block diagram illustrating a side view of the sport ball sanitizer 100 of FIG. 3 is shown. As may be seen from FIG. 4, both germicidal light sources 101 are horizontally oriented. In contrast, FIG. 5 illustrates an alternate embodiment in which one of the germicidal light sources 101 is horizontally oriented and the other germicidal light source 101 is vertically oriented midway between the two ends of the container 106. FIG. 6 illustrates an alternate embodiment that is wider than the other embodiments illustrating a capacity to simultaneously sanitize six balls 132.

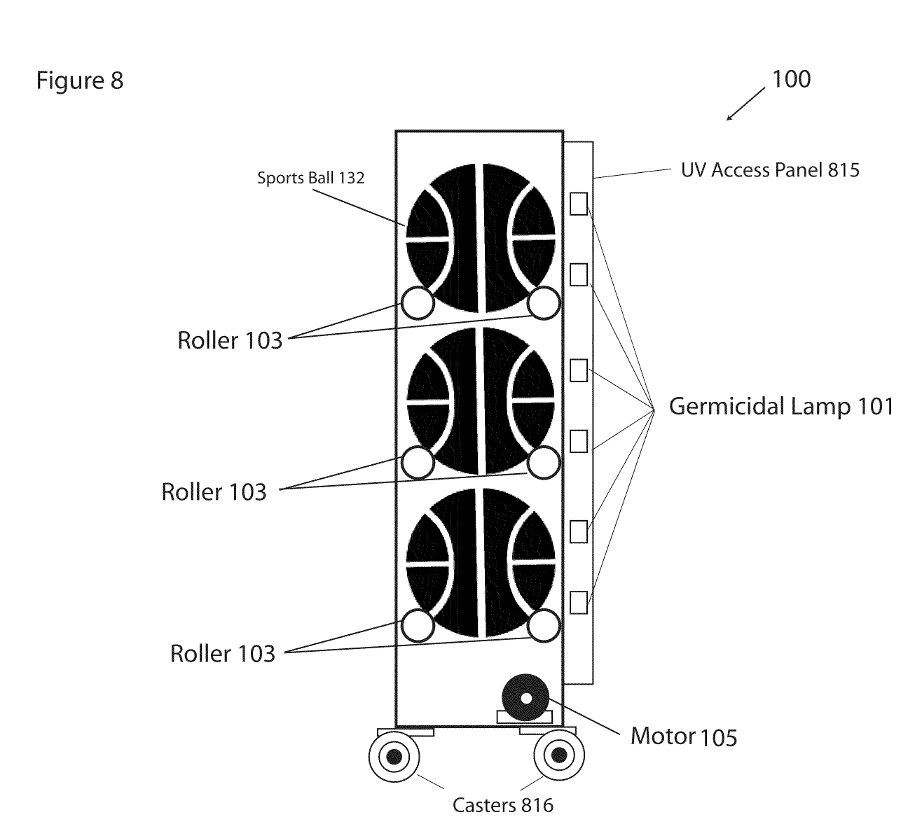

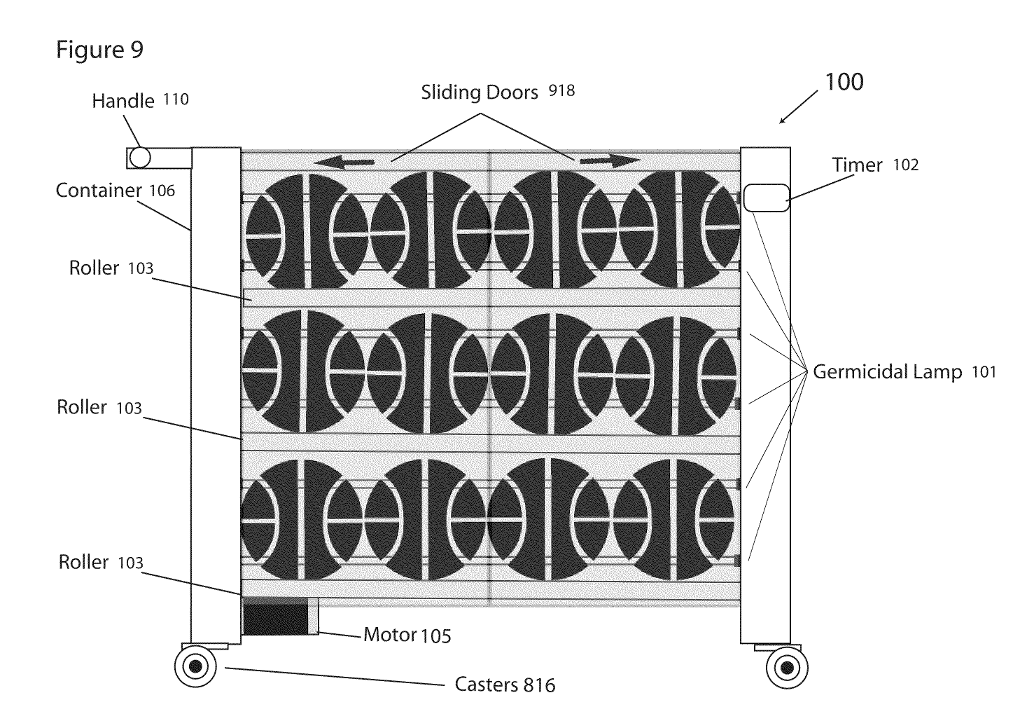

Referring now to FIG. 8, a block diagram illustrating an end view of a sport ball sanitizer 100 according to an alternate embodiment of the present invention is shown. The embodiment of FIG. 8 is similar to the embodiment shown in FIG. 1; however, the embodiment of FIG. 8 includes a plurality of rows of rollers 103 vertically stacked above one another for simultaneously sanitizing a plurality of rows of balls 132. The embodiment of FIG. 8 includes three rows of four balls 132 each, as shown in FIG. 9, which is a side view of the embodiment of FIG. 8. In the embodiment of FIG. 8, the motor 105 is in communication with at least one roller 103 in each row. In one embodiment, a gear (not shown) is coupled to the motor 105 and a gear (not shown) is coupled to each of the rollers 103. A chain engages each of the gears such that the motor 105 turns the chain which in turn causes the rollers 103 to rotate. In one embodiment, a serpentine belt is employed instead of the chain. FIG. 9 shows sliding doors 918 that may be opened to insert the balls. When the user opens the door, the kill switch 108 causes the germicidal light source 101 to stop emitting the germicidal light.

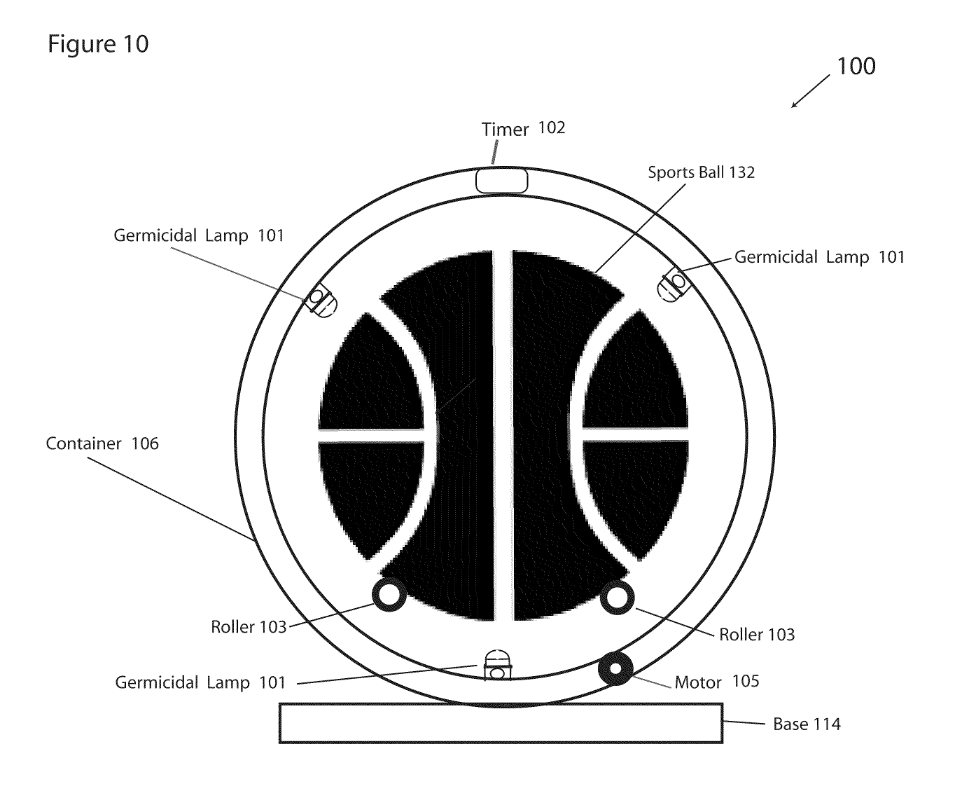

Referring now to FIG. 10, a block diagram illustrating an end view of a sport ball sanitizer 100 according to an alternate embodiment of the present invention is shown. The embodiment of FIG. 10 is different from the embodiment of FIG. 1 in that it provides a cylindrical container 106 that houses the other elements of the sport ball sanitizer 100. An advantage of the cylindrical container 106 is that it may provide a more uniform exposure of the ball 132 surface to the germicidal light, particularly in embodiments that include the reflective material and/or a plurality of germicidal light sources 101.

Referring now to FIG. 11, a block diagram illustrating an end view of a sport ball sanitizer 100 according to an alternate embodiment of the present invention is shown. Differences between the sport ball sanitizer 100 of FIG. 11 and the embodiments of FIGS. 1 through 10 are that the sport ball sanitizer 100 of FIG. 11 does not include a motor 105 or rollers 103. That is, the sport ball sanitizer 100 of FIG. 11 does not require rotation of the ball 132 in order to expose all locations of the ball 132 to the germicidal light for the required intensity-time. Instead, the sport ball sanitizer 100 of FIG. 11 includes a germicidal light-transparent receptacle 1125 that receives and supports the ball 132 to enable the germicidal light from the germicidal light source 101 to reach all locations on the surface of the ball 132. In one embodiment, the germicidal light-transparent receptacle 1125 comprises a glass crystal tube 1125, as shown in FIG. 11. However, the germicidal light-transparent receptacle 1125 may be constructed from other germicidal light-transparent materials and may be other than tubular-shaped. For example, the germicidal light-transparent receptacle 1125 may comprise a piece of bowl-shaped, or concave, germicidal light-transparent material upon which the user places the ball. The germicidal light-transparent receptacle 1125 may extend outside the container 106 by a mechanical arm so that the user may place the ball onto it, and then the arm may be retracted back into the container 106. An advantage of a germicidal light-transparent receptacle 1125 that encloses the ball 132 entirely, such as the tubular-shaped receptacle 1125, is that it may protect the germicidal light sources 101 from damage by the ball 132. In one embodiment, the sport ball sanitizer 100 of FIG. 11 includes a plurality of germicidal light sources 101 to generate a relatively uniform intensity of the germicidal light upon the ball 132, including one or more germicidal light source 101 on the door 1127 of the container 106, as shown in FIG. 11, and one or more germicidal light source 101 on the opposite end of the sport ball sanitizer 100, as shown in the side view of FIG. 12. In an alternate embodiment, the sport ball sanitizer 100 of FIG. 11 includes a single germicidal light source 101 and reflective material within all the interior surfaces of the container 106 to reflect the germicidal light to all locations on the surface of the ball 132. The container 106 mounted on a base 114 with a pivot 1126 to enable the user to easily remove the ball 132 by tilting the container 106, as shown. An advantage of the embodiment of FIG. 11 is that in some designs it may be less expensive because it does not incur the cost of a motor 105 and rollers 103. Another advantage is that it may have a longer mean time between failure due to the lack of moving parts.

Although the embodiments described herein are not limited to the following types of sport balls to be sanitized, examples of a sport ball 132 that may be sanitized by the embodiments described herein are a basketball, volleyball, football, soccer ball, rubber playground ball, kick ball, bowling ball, and baseball.

As may be observed from the foregoing, the amount of time required for the sport ball sanitizer 100 to operate to sanitize the ball 132 is a function of multiple factors, including but not limited to: the target germ set and desired kill rate, which dictate the required intensity-time; the number, location, and intensity rating of the germicidal light source(s) 101; the efficiency and location within the container 106 of the reflective material, if any; the distance of each location on the ball to the germicidal light source(s) 101, which is a function of the position of the germicidal light source(s) 101 within the container 106 and the shape and dimensions of ball 132; and the rotation speed and directions of the ball 132, if any. This amount of time required for sanitization may be calculated as a function of these design factors.

The present invention should not be limited by any of the exemplary embodiments described herein, but should be defined only in accordance with the following claims and their equivalents. Finally, those skilled in the art should appreciate that they can readily use the disclosed conception and specific embodiments as a basis for designing or modifying other structures for carrying out the same purposes of the present invention without departing from the scope of the invention as defined by the appended claims.

I claim:

1. An apparatus for sanitizing a sport ball, comprising:
a container, configured to receive the sport ball; and
at least one germicidal light source, coupled to the container, wherein the at least one germicidal light source is configured to emit germicidal wavelength light onto the sport ball within the container during operation of the apparatus.

2. The apparatus of claim 1, further comprising:
a timer, coupled to the container, configured to cause the apparatus to operate a predetermined amount of time.

3. The apparatus of claim 2, wherein the predetermined amount of time is selected to cause all locations on the surface of the ball to be exposed to the germicidal wavelength light at least a predetermined intensity-time.

4. The apparatus of claim 3, wherein the predetermined intensity-time is sufficient to achieve a predetermined kill rate for a target germ set.

5. The apparatus of claim 1, wherein the germicidal wavelength of the light is less than 300 nanometers.

6. The apparatus of claim 1, wherein the germicidal wavelength of the light is in a range between 240 and 280 nanometers.

7. The apparatus of claim 1, wherein the germicidal wavelength of the light is approximately 254.7 nanometers.

8. The apparatus of claim 1, wherein the container includes reflective material on at least a portion of its interior to reflect the germicidal wavelength light onto the surface of the sport ball.

9. The apparatus of claim 1, wherein the apparatus is configured to simultaneously sanitize a plurality of sport balls during operation.

10. The apparatus of claim 1, further comprising:
a rotation device, coupled to the container, wherein the rotation device is configured to rotate the ball to cause each location on the surface of the ball to be exposed to the germicidal wavelength light.

11. The apparatus of claim 10, wherein the rotation device comprises a motor.

12. The apparatus of claim 11, wherein the rotation device comprises one or more rollers, in communication with the motor, wherein the one or more rollers are configured support the ball and to be rolled by the motor to rotate the ball to cause each location on the surface of the ball to be exposed to the germicidal wavelength light.

13. The apparatus of claim 12, wherein the one or more rollers include helix activators to cause the ball to rotate in both the horizontal and vertical direction.

14. The apparatus of claim 1, further comprising:
a germicidal light-transparent receptacle, configured to receive and support the sport ball within the container, wherein the germicidal light source is configured to emit the germicidal wavelength light through the germicidal light-transparent container onto the sport ball.

15. A method for sanitizing a sport ball, the method comprising:
receiving the sport ball into a container; and
emitting germicidal wavelength light onto the sport ball within the container, wherein the germicidal wavelength light is emitted by a germicidal light source coupled to the container.

16. The method of claim 15, performing said emitting a predetermined amount of time.

17. The method of claim 16, wherein the predetermined amount of time is selected to cause all locations on the surface of the ball to be exposed to the germicidal wavelength light at least a predetermined intensity-time.

18. The method of claim 17, wherein the predetermined intensity-time is sufficient to achieve a predetermined kill rate for a target germ set.

19. The method of claim 15, further comprising:
rotating the ball to cause each location on the surface of the ball to be exposed to the germicidal wavelength light.

20. The method of claim 15, further comprising:
supporting the sport ball on a germicidal light-transparent receptacle, wherein said emitting the germicidal wavelength light comprises emitting the germicidal wavelength light through the germicidal light-transparent container onto the sport ball.

21. An apparatus for sanitizing a sport ball, comprising:
a germicidal light-transparent receptacle, configured to receive the sport ball; and
a plurality of germicidal light sources, coupled to the germicidal light-transparent receptacle, wherein the plurality of germicidal light sources are configured to emit germicidal wavelength light, at least in part through the germicidal light-transparent receptacle, onto each location on the surface of the ball for at least a predetermined intensity-time.
